(19) United States
(12) Patent Application Publication
(10) Pub. No.: US 2025/0009670 A1
(43) Pub. Date: Jan. 9, 2025

(54) Stable Solid Oral Formulation Of Tafamidis Or Pharmaceutically Acceptable Salt Thereof

(71) Applicant: Navinta, LLC, Ewing, NJ (US)

(72) Inventors: Niyaz Sultanbhai Mansuri, Ahmedabad (IN); Mahendra R. Patel, Delray Beach, FL (US); Bhavik Rajendrakumar Patel, Alladra (IN); Kartik Pandya, Vadodara (IN)

(21) Appl. No.: 18/734,639

(22) Filed: Jun. 5, 2024

Related U.S. Application Data
(60) Provisional application No. 63/471,117, filed on Jun. 5, 2023.
(60) Provisional application No. 63/627,890, filed on Feb. 1, 2024.
(60) Provisional application No. 63/627,892, filed on Feb. 1, 2024.

(51) Int. Cl. A61K 9/48; A61K 31/423

(52) U.S. Cl. CPC A61K 9/4866; A61K 9/4858; A61K 31/423

(57) ABSTRACT

The present invention relates to a solid oral powder filled hard gelatin capsule formulation of tafamidis, a pharmaceutically acceptable salt thereof, or tafamidis co-crystal(s), which involves use of standard dry blending ingredients and provides an improved dissolution profile. The formulation includes an effective amount of tafamidis, a pharmaceutically acceptable salt thereof, or a tafamidis co-crystal(s), at least one solubilizing agent; and at least one excipient selected from diluent, disintegrant and lubricant.

### FIELD OF THE INVENTION

The present invention relates to a pharmaceutically acceptable solid oral formulation of Tafamidis or a pharmaceutically acceptable salt(s) thereof or tafamidis co-crystal(s). Particularly, the present invention relates to an economically feasible and commercially viable pharmaceutically acceptable solid oral formulation of Tafamidis or a pharmaceutically acceptable salt(s) thereof or tafamidis co-crystal(s). More particularly, the present invention relates to a solid oral powder filled hard gelatin capsule formulation of Tafamidis or Tafamidis meglumine or Tafamidis co-crystal(s), which involves use of standard dry blending ingredients, wherein the formulation provides a excellent dissolution profile and stability.

### BACKGROUND OF THE INVENTION

Tafamidis (Brand VYNDAMAX®) and Meglumine salt of Tafamidis (Brand: VYNDAQEL®) in soft gelatin capsule dosage form have been approved by USFDA in 2019 for the treatment of the cardiomyopathy of wild type or hereditary transthyretin-mediated amyloidosis in adults. Chemically, Tafamidis is known as 2-(3,5-dichlorophenyl)-1,3-benzoxazole-6-carboxylic acid and Tafamidis meglumine is known as 2-(3,5-Dichlorophenyl)-1,3-benzoxazole-6-carboxylic acid-1-deoxy-1-(methyl-amino)-D-glucitol. The structures of Tafamidis (Formula-I) and Tafamidis meglumine (Formula-II) are as follows:

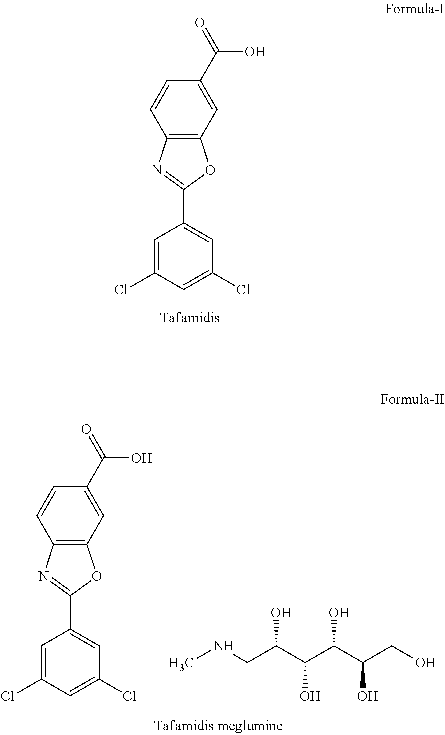 [SMILES: O=C(O)c1ccc2nc(-c3cc(Cl)cc(Cl)c3)oc2c1 ; O=C(O)c1ccc2nc(-c3cc(Cl)cc(Cl)c3)oc2c1 ; CNC[C@H](O)[C@@H](O)[C@H](O)[C@H](O)CO]

Tafamidis capsules 61 mg (VYNDAMAX®, Pfizer) is approved and available as a soft gelatin capsule. It is administered through oral route for the treatment of cardiomyopathy.

The active pharmaceutical ingredient (API) Tafamidis has a low aqueous solubility (BCS class-IV compound) and is provided in the form of suspended particles in an oily vehicle in the approved soft gelatin formulation.

Tafamidis meglumine capsules 20 mg (Vyndaqel®, Pfizer) is approved and is also available as a soft gelatin capsule. It is administered through oral route for the treatment of cardiomyopathy. Formulation of Tafamidis meglumine capsule also contains similar excipients as Tafamidis formulation and utilizes complex process to manufacture soft gelatin capsules.

According to the VYNDAQEL® European Public assessment report, “during development it was shown that tafamidis active substance, when formulated in a hard gelatin capsule with standard excipients, formed a rigid gel structure upon contact with aqueous vehicles thereby affecting the dissolution rate from the solid dosage.” The VYNDAQEL® European Public assessment report further states that “the chosen excipients in the capsule fill solution are: polyethylene glycol (suspending vehicle), sorbitan monoleate (surfactant/wetting agent), polysorbate 80 (surfactant/wetting agent),” while “the excipients in the capsule shell are: gelatin, propylene glycol, purified water, ‘Sorbitol special-glycerin blend’ (d-sorbitol, 1,4 sorbitan, mannitol and glycerin) and titanium dioxide (E 171) and black printing ink.”

The available soft gelatin formulation is associated with critical process requirements and requires specialized equipment set up. The soft gelatin formulation is relatively complex as compared to powder filled hard gelatin capsule dosage form. It is less preferred due to its complexity, manufacturing process/steps and equipment requirements. The manufacturing process for the soft gelatin capsule generally consists of mixing, homogenizing, de-aerating, encapsulation, drying, washing, in-line printing and packaging.

Dissolution performance for the reference listed drug (RLD) soft gelatin capsule (VYNDAMAX®) was identified as a major formulation hurdle (i.e., slow dissolution), for which a suspension-filled capsule was targeted for development. For the liquid suspension, a complex formulation was used, which included a base excipient that readily dispersed in aqueous media, a surfactant to promote dispersion and dissolution of the active substance in the formulation, a viscosity enhancing agent to promote suspension homogeneity during manufacture, and an antioxidant to minimize the risk of oxidative degradation and gelatin cross-linking. PEG 400 was used as the water miscible liquid base to promote rapid dispersion of Tafamidis suspension, following mixing with aqueous media during dissolution. Polysorbate 20 was selected as the surfactant based on the degree to which it is miscible with PEG 400 and able to maintain a homogeneous bulk fill. In combination, PEG 400 and polysorbate 20 constitute a base to which other excipients were added and assessed separately in prototypes manufactured exclusively for stability. Povidone K-90 was included to enhance viscosity in PEG-based liquids and reduce settling during manufacture. The soft gelatin capsule formulation available on the market is sensitive to higher temperature storage and results in significant yield loss due to cumbersome processing requirements.

U.S. Pat. No. 11,523,993 discloses dosage forms of tafamidis and its pharmaceutically acceptable salt, including both tablet and hard gelatin capsule compositions. The tablet composition comprises Tafamidis or its salt, at least one acidifier, and at least one pharmaceutically acceptable excipient. The hard gelatin capsule composition comprises spray dried or evaporated composition comprising at least one solubility enhancer, at least one disintegrant and at least one pharmaceutically acceptable excipient. The patent discusses the issues of formation of rigid gels in powder filled hard capsule formulations of tafamidis and tafamidis meglumine and states that the presence of an acidifier in the hard gel capsule formulation is necessary to inhibit gel formation during the dissolution. The patent further emphasizes the use of granulation and spray drying or evaporating methods, which are complex, time consuming and comparatively expensive.

U.S. Pat. Pub. No. 2023/0149365 discloses hard gelatin or HPMC capsule or tablet formulations of Tafamidis or Tafamidis meglumine that do not form rigid gels upon contacting with water or buffer solution. The publication describes hard gelatin or HPMC capsule formulations having easily dispersible granules and comprising at least one acidifier, as well as hard gelatin or HPMC capsule formulations having a spray-dried or evaporated composition.

WO 2024/084362 discloses soft gel capsule formulations containing crystalline forms of Tafamidis, in particular Tafamidis Form 6, and methods for their preparation. The publication describes solubility problems with soft gel capsule formulations of Tafamidis and the objective of providing a soft gel capsule formulation of Tafamidis that is stable and is prepared by the specific disclosed methods, i.e., the process of granulation using fluid bed granulator (top-spray granulation), which is complex and time consuming.

US Pat. Pub. No. 2022/0370365 discloses powdered formulations of tafamidis, wherein the formulations contain a carrier selected from magnesium aluminometasilicate, microcrystalline cellulose, low substituted hydroxypropyl cellulose, crospovidone and calcium silicate. The publication discusses disadvantages of soft gel capsule formulations of tafamidis and states that the disclosed invention overcomes these disadvantages by providing a powdered preparation that is optimal for tablet preparation. The publication further states that using the specifically disclosed carriers as adsorption carriers is necessary to suppress the decrease in the elution rate of tafamidis from the powdered preparation to achieve the objective of the disclosed invention.

U.S. Pat. No. 11,795,152 discloses crystalline forms of tafamidis such as Tafamidis fumaric acid co-crystal (named Form CSV), Tafamidis glutaric acid co-crystal (named Form CSVI) and Tafamidis adipic acid co-crystal (named Form CSVII). It further discloses pharmaceutical composition comprising a therapeutically effective amount of Form CSV, Form CSVI or Form CSVII and pharmaceutically acceptable carriers or excipients such as dibutylhydroxytoluene, polysorbate.

U.S. Pat. No. 11,878,081 discloses pharmaceutical unit-dose formulations suitable for oral administration that contain from 1% w/w to 20% w/w of a tafamidis-organic acid (such as adipic acid, glutaric acid, or fumaric acid) co-crystal(s), organic acid dissolution enhancer, polyethylene glycol, and polysorbate.

U.S. Pat. Pub. No. US2023/0149366 discloses a solid oral dosage form comprising tafamidis or a pharmaceutically acceptable salt or its solid-state forms or polymorphic forms thereof; at least one acidifier in an amount of about 0% w/w to about 10% w/w based on the total weight of the dosage form at least one pharmaceutically acceptable excipient. It further emphasizes the use of granulation and spray drying or evaporating methods.

Hence there is a need to develop alternative solid oral capsule formulations of Tafamidis or Tafamidis meglumine or Tafamidis co-crystal(s), which are simple and easy to manufacture, cost effective and commercially viable at large scale. It is also desirable to develop a capsule formulation that is smaller in size for ease of administration and to ensure better patient compliance. It is a further objective of the present invention to develop hard shell capsule formulations of Tafamidis and Tafamidis meglumine that do not form rigid gels upon contacting with water or buffer solution, and also do not require spray-dried or evaporation methods and/or the use of acidifiers. Yet further, it is desirable to develop a capsule formulation that includes a larger proportion of the active—Tafamidis or a pharmaceutically acceptable salt—to avoid the need for a patient to take multiple capsules at the same time to deliver a necessary dose of the active.

The present novel solid oral capsule formulations of Tafamidis or a pharmaceutically acceptable salt(s) thereof or Tafamidis co-crystal(s) fulfill the above-mentioned objectives.

### SUMMARY OF THE INVENTION

In one embodiment of the present invention, an oral pharmaceutically acceptable formulation includes:
an effective amount of tafamidis or a pharmaceutically acceptable salt(s) thereof or a tafamidis co-crystal(s);
at least one solubilizing agent; and
at least one excipient selected from diluent, disintegrant and lubricant;
wherein the formulation is provided in a form of a powder filled hard gelatin capsule

In some embodiments, the effective amount of tafamidis is in a range from about 50 mg/capsule to about 70 mg/capsule. In some of these embodiments, the effective amount of tafamidis is about 61.0 mg/capsule.

In some embodiments, the effective amount of tafamidis co-crystal(s) may be equivalent to tafamidis about 61.0 mg/capsule. In some embodiments, the tafamidis co-crystal(s) may include, but not limited to fumaric acid, adipic acid, glutaric acid, or a mixture thereof.

In some embodiments, the pharmaceutically acceptable salt of tafamidis is tafamidis meglumine in an amount from about 20 mg/capsule to about 80 mg/capsule. In certain of these embodiments, tafamidis meglumine is present in an amount of about 20 mg/capsule, or in an amount of 80 mg/capsule.

In some embodiments, the at least one solubilizing agent is selected from cyclodextrins, substituted cyclodextrin derivatives, and mixtures thereof. In certain of these embodiments, the at least one solubilizing agent is hydroxypropyl β-cyclodextrin.

In some embodiments, the at least one solubilizing agent is provided in a range from about 10 wt. % to about 75 wt. %, or from about 20 wt. % to about 60 wt. %, or from about 25 wt. % to about 40 wt. %.

In certain embodiments, the at least one diluent is selected from lactose, pregelatinized starch, corn starch, microcrystalline cellulose, mannitol, dicalcium phosphate, and mixtures thereof.

In some embodiments, the at least one diluent is provided in a range from about 3 wt. % to about 60 wt. %, from about 7.5 wt. % to about 50 wt. %, or from about 12 wt. % to about 40 wt. %.

In some embodiments, the at least one disintegrant is selected from sodium starch glycolate, crospovidone, croscarmellose sodium, and mixtures thereof.

In certain embodiments, the at least one disintegrant is provided in a range from about 1 wt. % to about 10 wt. %, from about 1 wt. % to about 5 wt. %, or from about 1 wt. % to about 3 wt. %.

In some embodiments, the at least one lubricant is selected from sodium stearyl fumarate, talc, magnesium stearate, and mixtures thereof.

In some embodiments, the at least one lubricant is provided in a range from about 0.5 wt. % to about 5 wt. %, from about 0.5 wt. % to about 3 wt. %, or from about 1 wt. % to about 2 wt. %.

A method of treating a transthyretin amyloid disease in mammals is also provided, the method including the steps of administering a therapeutically effective amount of the pharmaceutically acceptable capsule formulation described herein to a mammal in need thereof.

In some embodiments, the powder filled hard gelatin capsule is prepared by a dry blending process.

In some embodiments, the powder filled hard gelatin capsule is prepared by blending (physical mixture) of solubilizing agent and Tafamidis or Tafamidis meglumine or Tafamidis co-crystal(s), followed by blending with other excipients.

In some embodiments, the powder filled hard gelatin capsule is prepared by griding of solubilizing agent and Tafamidis or Tafamidis meglumine or tafamidis co-crystal(s), followed by blending with other excipients.

In another embodiment of the present invention, the solid oral pharmaceutical preparation of Tafamidis or Tafamidis meglumine or tafamidis co-crystal(s) provides a faster dissolution profile than the RLD.

In a further embodiment of the present invention, the solid oral preparation of Tafamidis or Tafamidis meglumine or tafamidis co-crystal(s) is prepared by following method:
an aqueous paste of at least one solubilizing agent is prepared;
Tafamidis or Tafamidis meglumine or tafamidis co-crystal(s) is added to the aqueous paste under continuous kneading;
a resultant paste is dried, sized and mixed with other excipients to fill as powder in hard gelatin capsule shell.

In yet another embodiment, the present invention relates to a non-liquid suspension solid dosage form of tafamidis or its salt(s) or its co-crystal(s), which is powder filled hard gelatin capsule and it is bioequivalent of RLD.

Other features and advantages of the present invention will become apparent from the following more detailed description, which illustrates, by way of example, the principle of the invention.

### BRIEF DESCRIPTION OF THE DRAWINGS

FIG. 1 is XRPD of Tafamidis API before prior to its use in formulation.

FIG. 2 is XRPD of Tafamidis API from formulation in Example 1 (at stress condition, 50° C., 1 week, open) in accordance with the present invention.

FIG. 3 is XRPD of a placebo of Tafamidis formulation (at stress condition, 50° C., 1 week, open).

FIG. 4 is XRPD of Tafamidis meglumine API prior to its use in formulation.

FIG. 5 is XRPD of Tafamidis meglumine API from formulation in Example 3 at accelerated condition 40° C.±2° C./75% RH±5% RH, 3 Months) in accordance with the present invention.

FIG. 6 is XRPD of a placebo of Tafamidis meglumine formulation (at accelerated condition 40° C.±2° C./75% RH±5% RH, 3 Months).

FIG. 7 shows dissolution profile of tafamidis hard gelatin 61 mg capsule in accordance with the present invention.

FIG. 8 shows dissolution profile of tafamidis meglumine hard gelatin 20 mg capsule in accordance with the present invention.

FIG. 9 shows dissolution profile of tafamidis 61 mg capsule in accordance with the present invention.

FIG. 10 shows dissolution profile of tafamidis meglumine 80 mg capsule in accordance with the present invention and RLD.

FIG. 11 shows dissolution profile of tafamidis meglumine 20 mg capsule in accordance with the present invention.

FIG. 12 shows dissolution profile of tafamidis hard gelatin 61 mg capsule in accordance with the present invention.

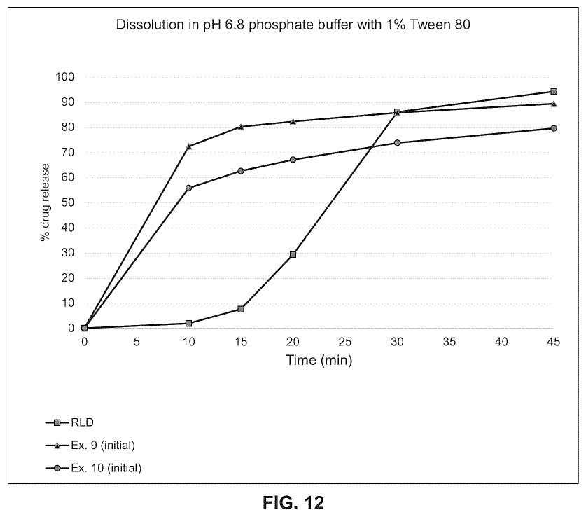

FIG. 13 shows dissolution profile of tafamidis meglumine hard gelatin 80 mg capsule in accordance with the present invention.

### DETAILED DESCRIPTION OF THE INVENTION

The term “drug” or “active ingredient” refers to an agent, active ingredient compound or other substance, or compositions and mixture thereof that provide some pharmacological, often beneficial, effect. Reference to a specific active ingredient shall include where appropriate the active ingredient and its pharmaceutically acceptable salts.

The term “dosage form” refers to any form of the formulation, e.g., a tablet or capsule or parenteral, which comprises a pharmaceutically effective amount of an active ingredient sufficient to achieve a therapeutic effect and contains other excipients.

The term “co-crystal(s)” refers to a single-phase crystalline solid material composed of at least two different molecular and/or ionic compounds that are neither solvates nor simple salts and that exist in a stoichiometric ratio.

It is noted that, as used in the specification and the claims, the singular form “a,” “an,” and “the” comprises plural referents unless the context clearly indicates otherwise. For example, reference to a component in the singular is intended to comprise a plurality of components.

The term “about” is to be construed as modifying a term or value such that it is not an absolute. This term will be defined by the circumstances.

This includes, at the very least, the degree of expected experimental error, technique error and instrument error for a given technique used to measure a value. In general, this term used in connection with a numerical value throughout the specification and the claims denotes an interval of accuracy, familiar and acceptable to a person skilled in the art. In general, such interval of accuracy is ±10%. Thus, “about ten” means 9 to 11. All numbers in this description indicating amounts, ratios of materials, physical properties of materials, and/or use are to be understood as modified by the word “about,” except as otherwise explicitly indicated.

The term “comprising” is synonymous with “including,” “having,” “containing,” or “characterized by.” These terms are inclusive and open-ended and do not exclude additional, unrecited elements or method steps.

The term “polymorph” refers to different crystalline forms of the same compound and includes, but is not limited to, other solid state molecular forms including hydrates (e.g., bound water present in the crystalline structure) and solvates (e.g., bound solvents other than water) of the same compound.

The term “stable” as used herein refers to physical stability, chemical stability, and polymorphic stability of a solid oral dosage form. Physical stability refers to consistent physical properties of composition throughout the product self-life, e.g., suspension/solution appearance, description, consistency, flow properties, manufacturability, and others. Chemical stability refers to consistency in obtaining acceptable results of drug assay, drug content uniformity and drug related substances. Polymorphic stability refers to retention polymorphic type of Tafamidis API in the finished product and no polymorph conversions throughout the product self-life for e.g., at initial and at accelerated stability conditions (40° C./75% RH 6 months). The different physical properties exhibited by different solid forms of a pharmaceutical compound can affect important pharmaceutical parameters such as storage, stability, compressibility, density (important in formulation and product manufacturing), and dissolution rates (important in determining bioavailability). Stability differences may result from changes in chemical reactivity (e.g., differential hydrolysis or oxidation, such that a dosage form comprising a certain polymorph can discolor more rapidly than a dosage form comprising a different polymorph), mechanical changes (e.g., tablets can crumble on storage as a kinetically favored crystalline form converts to thermodynamically more stable crystalline form), or both (e.g., tablets of one polymorph can be more susceptible to breakdown at high humidity). Solubility differences between polymorphs may, in extreme situations, result in transitions to crystalline forms that lack potency and/or that are toxic. In addition, the physical properties of a crystalline form may also be important in pharmaceutical processing.

The term “water-soluble excipients” refers to compounds which are freely soluble in water. Some preferred water-soluble excipients may be cyclodextrin or its derivatives, inorganic ionic compounds, and carbohydrate compounds. The water-soluble excipients may be dissolved in the 900 mL of dissolution medium.

The term “inorganic ionic compound” refers to a compound which has positive and negative ions, is soluble in water and increases the wetting/channeling of a powder mixer in dry form or wet form.

The term “carbohydrate” refers to sugar compounds that are soluble in water and increase the channeling of the powder mixer in dry form or wet form.

The term “solubilizing agent” refers to compound/surfactants, which increases the solubility of one agent in another.

The term “lubricant” refers to agents added to tablet and capsule formulations in a very small quantity (usually around 0.25%-5.0% wt.) to improve powder processing properties of formulations.

The term “diluent” refers to a substance that increases the size of the dosage form. Diluents are usually added to tablets or capsules where the active constituent is in low dose and to improve the powder flow and compaction properties prior to direct compression. Diluents are commonly used in the range between about 5% and 80% wt.

The term “disintegrant” refers to the substance, which is an additive that promotes disintegration, which is the breakage of a tablet or capsule into small fragments when in contact with a liquid medium.

The term “solid dosage form” refers to a solid pharmaceutical preparation in the form of, for example, a tablet, capsule, pill, powder, or granule.

The present invention provides pharmaceutically acceptable solid oral formulation of Tafamidis or a pharmaceutically acceptable salt thereof or Tafamidis co-crystal(s). Particularly, the present invention relates to an economically feasible and commercially viable pharmaceutically acceptable solid oral capsule formulation of Tafamidis or a pharmaceutically acceptable salt thereof or Tafamidis co-crystal(s) prepared with water-soluble excipients. More particularly, the present invention relates to a solid oral powder filled hard gelatin capsule formulation of Tafamidis or a pharmaceutically acceptable salt thereof or Tafamidis co-crystal(s) that uses standard dry blending ingredients to provide a similar or faster dissolution profile than the RLD.

The present inventors have discovered unexpectedly that a simple capsule formulation using standard dry ingredients provided a faster dissolution profile than the RLD. The developed hard gelatin powder fill capsule formulation is comparable in size or smaller than the soft gelatin capsule formulation of the RLD, which makes it easier for administration and improves the patient compliance.

Alternatively, the inventive hard gelatin powder fill capsule formulations may be provided in a comparable size but having a much higher dose of the active (Tafamidis or a pharmaceutically acceptable salt thereof), such that the number of capsules needed to be ingested to provide a desirable amount of the active agent may be reduced. This also makes it easier for administration and improves the patient compliance.

In one embodiment of the present invention, a pharmaceutically acceptable capsule formulation includes:
an effective amount of tafamidis or tafamidis meglumine or tafamidis co-crystal(s); and
an effective amount of one or more water-soluble excipients.

In some embodiments, the water-soluble excipients include, but are not limited to, cyclodextrin or its derivatives, inorganic ionic compounds, carbohydrates, or a mixture thereof. The water-soluble excipients may be provided in a range of about 30% wt. to about 99.5% wt.

In one embodiment of the present invention, an oral pharmaceutically acceptable formulation of powder filled hard gelatin capsule comprising:
an effective amount of tafamidis or tafamidis meglumine or tafamidis co-crystal(s);
at least one solubilizing agent; and
at least one excipient selected from a diluent, a disintegrant and a lubricant.

In some embodiments, the effective amount of tafamidis may be in the range of about 50 mg/capsule to about 80 mg/capsule. In certain of those embodiments, the effective amount of tafamidis may be about 61.0 mg/capsule. In some embodiments, the effective amount of tafamidis co-crystals(s) may be equivalent to about 61 mg/capsule.

In some embodiments, the tafamidis co-crystal(s) may include, but are not limited to, fumaric acid, adipic acid, glutaric acid, or mixtures thereof. In one preferable embodiment, the tafamidis co-crystal is tafamidis fumaric acid co-crystal. Examples of Tafamidis co-crystals are described in U.S. Pat. No. 11,795,152 and 11,878,081, the disclosure of which is incorporated herein by reference.

In some embodiments, the effective amount of tafamidis meglumine may be in the range of about 10 mg/capsule to about 90 mg/capsule. In certain of those embodiments, the effective amount of Tafamidis meglumine may be about 20.0 mg/capsule. In additional embodiments, the effective amount of Tafamidis meglumine may be about 80.0 mg/capsule.

In some embodiments, the solubilizing agent is selected from cyclodextrins (i.e., cyclic oligosaccharides derived from starch) like α-, β-, and γ-cyclodextrin, and substituted cyclodextrin derivatives like hydroxypropyl β-cyclodextrin and sulfobutylether-β-cyclodextrin.

The solubilizing agent may be provided in a range (wt. %) from about 10 to about 75, preferably from about 20 to about 60, and more preferably about 25 to about 40.

The cyclodextrin and/or its derivative may be provided in a range from about 10% wt. to about 75% wt., preferably from about 20% wt. to about 60% wt., and more preferably from about 25% wt. to about 40% wt.

In some embodiments, the inorganic ionic compound may be selected from sodium chloride, calcium chloride, potassium chloride, lithium chloride or a mixture thereof.

The inorganic ionic compound may be provided in a range (wt. %) from about 10 to about 60, preferably from about 10 to about 50, and more preferably from about 10 to about 30.

In some embodiments, the carbohydrate compound may be selected from sucrose, sorbitol, mannitol, or a mixture thereof.

The carbohydrate compound may be provided in a range (wt. %) from about 10 to about 60, preferably from about 10 to about 50, and more preferably from about 10 to about 30.

In some embodiments, the capsule formulation further contains at least one excipient which may be selected from a diluent, a disintegrant and a lubricant.

The diluent may be selected from lactose, pregelatinized starch, corn starch, microcrystalline cellulose, mannitol, dicalcium phosphate, or mixtures thereof. In some embodiments, the formulation may be essentially free of, or free of, microcrystalline cellulose.

In some embodiments, the diluent is provided in a range (wt. %) from about 3 to about 60, preferably from about 7.5 to about 50, and more preferably about 12 to about 40.

The disintegrant may be selected from sodium starch glycolate (SSG), crospovidone, croscarmellose sodium, or mixtures thereof. In some embodiments, the formulation may be essentially free of, or free of, crospovidone.

The disintegrant may be provided in a range (wt. %) from about 1 to about 10, or from about 1 to about 5, or from about 1 to about 3.

In some embodiments, the lubricant is selected from sodium stearyl fumarate, talc, magnesium stearate, or mixtures thereof.

The lubricant may be provided in a range (wt. %) from about 0.5 to about 5, preferably from about 0.5 to about 3, and more preferably about 1 to about 2.

In some embodiments, the formulations disclosed herein may be free or substantially free of acidifiers, such as organic acids and inorganic acids.

In some embodiments, the inventive formulation may be a non-liquid suspension solid dosage form of tafamidis or its salt(s) or its co-crystal(s), wherein the dosage form is a powder filled hard gelatin capsule, which is bioequivalent to RLD.

The inventive formulations may be a solid oral powder filled hard gelatin capsule prepared with a dry blending process or wet granulation process. In some embodiments, the inventive formulations are prepared by dry blending process only. In some embodiments, the manufacturing process for the inventive formulations does not involve evaporation or spray-drying steps. In some embodiments, the formulation may be essentially free of, or free of, water. In some embodiments, the process of manufacturing the inventive formulations does not involve the use of water or another solvent.

The inventive formulation surprisingly exhibits a similar or improved dissolution profile as compared to the available RLD soft gelatin capsule formulation.

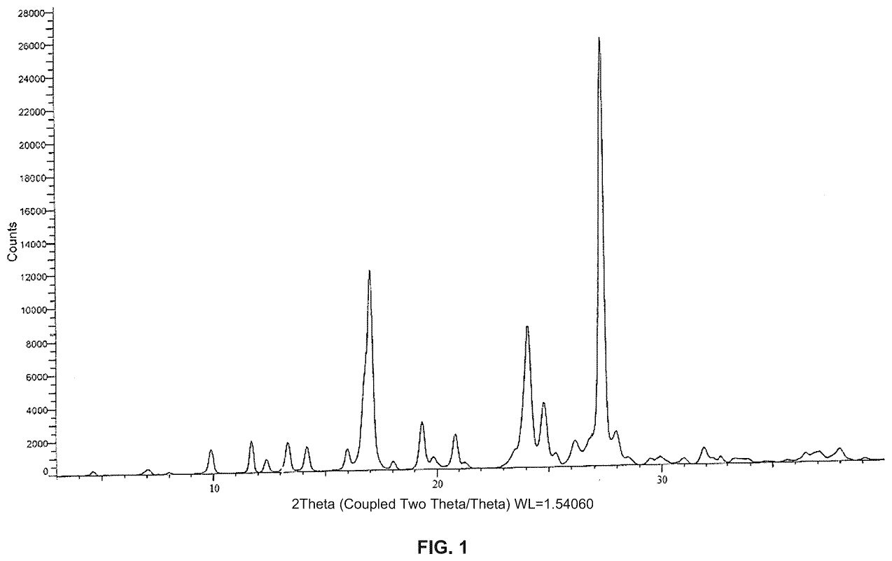

The inventive formulations are prepared by simple manufacturing process. The inventive formulation retains the original polymorphic form of Tafamidis in the formulation as supported by XRD analysis in FIGS. 1-5. The soft gelatin capsule formulations of RLD are difficult to manufacture, are very variable in dissolution results, involve a high cost of production and are prone chemical degradation and polymorphic conversion.

### EXPERIMENTAL

General Manufacturing process for the preparation of one exemplary pharmaceutically acceptable solid oral hard gelatin powder filled capsule formulation of the present invention is as follows:
a) raw material was dispensed as per batch composition;
b) a diluent was sifted using a vibratory sifter;
c) the material of step-b was loaded into a double cone blender and mixed;
d) Tafamidis or Tafamidis meglumine, or another pharmaceutically acceptable salt of tafamidis, or tafamidis co-crystal(s), another diluent and solubilizing agent were co-sifted using a vibratory sifter;
e) the material of step-d was loaded into the step-c in a double cone blender and mixed;
f) lubricant was separately sifted using a vibratory sifter;
g) the sifted lubricant was added into the step-e in a double cone blender and mixed; and
h) the material obtained in step-g was encapsulated in a hard gelatin capsule using a capsule filling machine.

General Manufacturing process for the preparation of an exemplary embodiment of pharmaceutically acceptable solid oral hard gelatin capsule formulation of the present invention:
a) Raw material was dispensed as per batch composition;
b) raw materials were sifted using a vibratory sifter;
c) Tafamidis or Tafamidis meglumine, or another pharmaceutically acceptable salt of tafamidis, or Tafamidis co-crystal(s), one or more solubilizing agents and one or more diluents were mixed in high shear mixer granulator;
d) the material of step-c was granulated with separately prepared solution of povidone in IPA, in rapid mixer granulator until a suitable granular wet mass was obtained;
e) the obtained wet mass was dried, sized and mixed further with other excipients in double cone blender;
f) lubricant was separately sifted using a vibratory sifter;
g) the lubricant was added into the step-e in a double cone blender and mixed; and
h) the material obtained in step-g was encapsulated in a hard gelatin capsule using a capsule filling machine.

Packaging of pharmaceutically acceptable formulation of Tafamidis or Tafamidis meglumine or Tafamidis co-crystal(s) of the present invention:

Filled capsules were packaged in the below pack configurations:
10's Aluminum blister pack, and
30's HDPE bottle pack.

### EXAMPLES

### Example 1: Preparation of Pharmaceutically Acceptable Hard Gelatin 61 mg Capsule of Tafamidis

| Sr. | | | |
|---|---|---|---|
| No. | Ingredients | mg/capsule | % w/w |
| 1 | Tafamidis | 61.00 | 26.52 |
| 2 | Pregelatinized Starch (Starch 1500) | 76.00 | 33.04 |
| 3 | Colloidal silicon dioxide (Aerosil 200) | 5.00 | 2.17 |
| 4 | Hydroxy propyl β-cyclodextrin | 80.00 | 34.78 |
| 5 | Sodium starch glycolate (Type A) | 5.00 | 2.17 |
| 6 | Sodium stearyl fumarate | 3.00 | 1.30 |
| Total fill content (mg) | | 230.00 | 100.00 |
| 7 | Hard gelatin capsule shell (Size 1) red | 76.00 | — |
| | opaque cap and red opaque body. | | |

| | | |
|---|---|---|
| Total weight of filled capsule (mg) | 306.00 | — |

### Example 2: Preparation of Pharmaceutically Acceptable Hard Gelatin 61 mg Capsule of Tafamidis

| Sr. | | | |
|---|---|---|---|
| No. | Ingredients | mg/capsule | % w/w |
| 1 | Tafamidis | 61.00 | 26.52 |
| 2 | Lactose monohydrate | 76.00 | 33.04 |
| 3 | Colloidal silicon dioxide (Aerosil 200) | 5.00 | 2.17 |
| 4 | Hydroxypropyl β-cyclodextrin | 80.00 | 34.78 |
| 5 | Sodium starch glycolate (Type A) | 5.00 | 2.17 |
| 6 | Sodium stearyl fumarate | 3.00 | 1.30 |
| Total fill content (mg) | | 230.00 | 100.00 |
| 7 | Hard gelatin capsule shell (Size 1) red | 76.00 | — |
| | opaque cap and red opaque body. | | |

| | | |
|---|---|---|
| Total weight of filled capsule (mg) | 306.00 | — |

### Example 3: Preparation of Pharmaceutically Acceptable Hard Gelatin 20 mg Capsule of Tafamidis Meglumine

| Sr. | | | |
|---|---|---|---|
| No. | Ingredients | mg/capsule | % w/w |
| 1 | Tafamidis Meglumine | 20.00 | 7.84 |
| 2 | Lactose monohydrate | 135.00 | 52.94 |
| 3 | Colloidal silicon dioxide (Aerosil 200) | 2.00 | 0.78 |
| 4 | Hydroxypropyl β-cyclodextrin | 90.00 | 35.29 |
| 5 | Sodium starch glycolate (Type A) | 5.00 | 1.96 |
| 6 | Sodium stearyl fumarate | 3.00 | 1.18 |
| Total fill content (mg) | | 255.00 | 100.00 |
| 7 | Hard gelatin capsule shell (Size 1) yellow | 76.00 | — |
| | opaque cap and yellow opaque body. | | |

| | | |
|---|---|---|
| Total weight of filled capsule (mg) | 331.00 | — |

### Example 4: Preparation of a Pharmaceutically Acceptable Hard Gelatin 61 mg Capsule of Tafamidis

| Sr. | | | |
|---|---|---|---|
| No. | Ingredients | mg/capsule | % w/w |
| 1 | Tafamidis | 61.00 | 26.6 |
| 2 | Pregelatinized starch | 31.00 | 13.47 |
| 3 | Colloidal silicon dioxide (Aerosil 200) | 5.00 | 2.17 |
| 4 | Hydroxypropyl β-cyclodextrin | 80.00 | 34.78 |
| 5 | Sodium Chloride | 45.00 | 19.56 |
| 6 | Sodium starch glycolate (Type A) | 5.00 | 2.17 |
| 7 | Sodium stearyl fumarate | 3.00 | 1.30 |
| Total fill content (mg) | | 230.00 | 100.00 |
| 8 | Hard gelatin capsule shell (Size 1) red | 76.00 | — |
| | opaque cap and red opaque body. | | |

| | | |
|---|---|---|
| Total weight of filled capsule (mg) | 306.00 | — |

### Example 5: Preparation of a Pharmaceutically Acceptable Hard Gelatin 61 mg Capsule of Tafamidis

| Sr. | | | |
|---|---|---|---|
| No. | Ingredients | mg/capsule | % w/w |
| 1 | Tafamidis | 61.00 | 26.6 |
| 2 | Sorbitol | 31.00 | 13.47 |
| 3 | Colloidal silicon dioxide (Aerosil 200) | 5.00 | 2.17 |
| 4 | Hydroxypropyl β-cyclodextrin | 80.00 | 34.78 |
| 5 | Sodium Chloride | 45.00 | 19.56 |
| 6 | Sodium starch glycolate (Type A) | 5.00 | 2.17 |
| 7 | Sodium stearyl fumarate | 3.00 | 1.30 |
| Total fill content (mg) | | 230.00 | 100.00 |
| 8 | Hard gelatin capsule shell (Size 1) red | 76.00 | — |
| | opaque cap and red opaque body. | | |

| | | |
|---|---|---|
| Total weight of filled capsule (mg) | 306.00 | — |

### Example 6: Preparation of Pharmaceutically Acceptable Hard Gelatin 20 mg Capsule of Tafamidis Meglumine

| Sr. | | | |
|---|---|---|---|
| No. | Ingredients | mg/capsule | % w/w |
| 1 | Tafamidis meglumine | 20.00 | 7.84 |
| 2 | Hydroxypropyl β-cyclodextrin | 90.00 | 35.29 |
| 3 | Pregelatinized starch | 130.00 | 50.98 |
| 4 | Sodium starch glycolate (Type A) | 5.00 | 1.96 |
| 5 | Povidone K-30 | 5.00 | 1.96 |
| 6 | Isopropyl alcohol | q.s. | — |
| 7 | Colloidal silicon dioxide | 2.00 | 0.78 |
| 8 | Sodium stearyl fumarate | 3.00 | 1.18 |
| Fill weight (mg) | | 255.00 | 100.00 |
| 8 | Hard gelatine capsule shell (Size 1) | 76.00 | — |
| | yellow opaque cap and yellow opaque | | |
| | body. | | |

| | | |
|---|---|---|
| Total weight of filled capsule (mg) | 306.00 | — |

### Example 7: Preparation of a Pharmaceutically Acceptable Hard Gelatin 61 mg Capsule of Tafamidis

| Sr. | | | |
|---|---|---|---|
| No. | Ingredients | mg/capsule | % w/w |
| 1 | Tafamidis | 61.00 | 22.59 |
| 2 | Hydroxypropyl β-cyclodextrin | 80.00 | 29.63 |
| 3 | Sodium Chloride | 45.00 | 16.67 |
| 4 | Pregelatinised starch | 68.50 | 25.37 |
| 5 | Povidone K30 | 5.00 | 1.85 |
| 6 | Water | q.s | q.s |
| 7 | Colloidal silicon dioxide (Aerosil 200) | 2.50 | 0.93 |
| 8 | Sodium starch glycolate (Type A) | 5.00 | 1.85 |
| 9 | Sodium stearyl fumarate | 3.00 | 1.11 |
| Total fill content (mg) | | 270.00 | 100.00 |
| 10 | Hard gelatin capsule shell (Size 1) red | 76.00 | — |
| | opaque cap and red opaque body. | | |

| | | |
|---|---|---|
| Total weight of filled capsule (mg) | 346.00 | — |

### Example 8: Preparation of Pharmaceutically Acceptable Hard Gelatin 80 mg Capsule of Tafamidis Meglumine

| Sr. | | | |
|---|---|---|---|
| No. | Ingredients | mg/capsule | % w/w |
| 1 | Tafamidis meglumine | 80.00 | 21.45 |
| 2 | Hydroxypropyl β-cyclodextrin | 263.00 | 70.51 |
| 3 | Pregelatinized starch | 12.00 | 3.22 |
| 4 | Sodium starch glycolate (Type A) | 5.00 | 1.34 |
| 5 | Povidone K-30 | 4.00 | 1.07 |
| 6 | Isopropyl alcohol | q.s. | 1 |
| 7 | Colloidal silicon dioxide | 5.00 | 1.34 |
| 8 | Sodium stearyl fumarate | 4.00 | 1.07 |
| Fill weight (mg) | | 373.00 | 100.00 |
| 9 | Hard gelatine capsule shell (Size 0el) | 104.00 | — |
| | yellow opaque cap and yellow opaque | | |
| | body. | | |

| | | |
|---|---|---|
| Total weight of filled capsule (mg) | 477.00 | — |

### Example 9: Preparation of Pharmaceutically Acceptable Hard Gelatin 72.49 mg Capsule of Tafamidis Fumaric Acid Co-Crystal

| Sr. | | mg/ | |
|---|---|---|---|
| No. | Ingredients | capsule | % w/w |
| 1 | Tafamidis fumaric acid co-crystal | 72.49 | 26.85 |
| 2 | Pregelatinized Starch (Starch 1500) | 57.01 | 21.11 |
| 3 | Colloidal silicon dioxide (Aerosil 200) | 2.50 | 0.93 |
| 4 | Hydroxy propyl β-cyclodextrin | 80.00 | 29.63 |
| 5 | Sodium Chloride | 45.00 | 16.67 |
| 6 | Sodium starch glycolate (Type A) | 5.00 | 1.85 |
| 7 | Polyvinyl pyrrolidone K-30 | 5.00 | 1.85 |
| 8 | Sodium stearyl fumarate | 3.00 | 1.11 |
| 9 | Purified water | q.s. | q.s. |
| Total fill content (mg) | | 270.00 | 100.00 |
| 10 | Hard gelatin capsule shell (Size 1) red | 76.00 | — |
| | opaque cap and red opaque body. | | |

| | | |
|---|---|---|
| Total weight of filled capsule (mg) | 346.00 | — |

### Example 10: Preparation of Pharmaceutically Acceptable Hard Gelatin 72.49 mg Capsule of Tafamidis Fumaric Acid Co-Crystal

| Sr. | | mg/ | |
|---|---|---|---|
| No. | Ingredients | capsule | % w/w |
| 1 | Tafamidis fumaric acid co-crystal | 72.49 | 26.85 |
| 2 | Pregelatinized Starch (Starch 1500) | 137.01 | 50.74 |
| 3 | Colloidal silicon dioxide (Aerosil 200) | 2.50 | 0.93 |
| 4 | Sodium Chloride | 45.00 | 16.67 |
| 5 | Sodium starch glycolate (Type A) | 5.00 | 1.85 |
| 6 | Polyvinyl pyrrolidone K-30 | 5.00 | 1.85 |
| 7 | Sodium stearyl fumarate | 3.00 | 1.11 |
| 8 | Purified water | q.s. | q.s. |
| Total fill content (mg) | | 270.00 | 100.00 |
| 9 | Hard gelatin capsule shell (Size 1) red | 76.00 | — |
| | opaque cap and red opaque body. | | |

| | | |
|---|---|---|
| Total weight of filled capsule (mg) | 346.00 | — |

### Example 11: Preparation of Pharmaceutically Acceptable Hard

| Sr. | | mg/ | |
|---|---|---|---|
| No. | Ingredients | capsule | % w/w |
| 1 | Tafamidis meglumine | 80 | 21.05 |
| 2 | Hydroxypropyl β-cyclodextrin | 240.00 | 63.16 |
| 3 | Pregelatinized starch | 12.00 | 3.16 |
| 4 | Sodium Chloride | 30.00 | 7.89 |
| 5 | Sodium starch glycolate (Type A) | 5.00 | 1.32 |
| 6 | Povidone K-30 | 4.00 | 1.05 |
| 7 | Isopropyl alcohol | q.s. | q.s. |
| 8 | Colloidal silicon dioxide | 5.00 | 1.32 |
| 9 | Sodium stearyl fumarate | 4.00. | 1.05 |
| Total fill content (mg) | | 380.00 | 100.00 |
| 10 | Hard gelatin capsule shell (Size 0el) yellow | 104.00 | — |
| | opaque cap and yellow opaque body. | | |

| | | |
|---|---|---|
| Total weight of filled capsule (mg) | 477.00 | — |

In some embodiments, the inventive formulation may utilize a micronized form of Tafamidis or Tafamidis meglumine API (e.g., D90 being less than 20 micron).

In some embodiments, the capsule formulation of tafamidis or a pharmaceutically acceptable salt thereof may include hydroxypropyl β-cyclodextrin as a key excipient used as solubilizing agent, which is expected to provide improved dissolution.

In some embodiments, the capsule formulation of tafamidis or a pharmaceutically acceptable salt thereof or tafamidis co-crystal(s) may contain hydroxypropyl β-cyclodextrin, sodium chloride, and a carbohydrate compound as key excipients, which are expected to provide an improved dissolution profile. In additional embodiments, the formulation may be free of sodium chloride and/or carbohydrates.

Dissolution results of the inventive formulations of Tafamidis and Tafamidis meglumine in comparison to the approved RLD soft gelatin capsule formulations are presented below in Tables 1-9. XRPD analysis results of inventive formulations, indicating retention of the original polymorphic form of Tafamidis and Tafamidis meglumine in the formulation are presented below in Table 10 and Table 11 respectively.

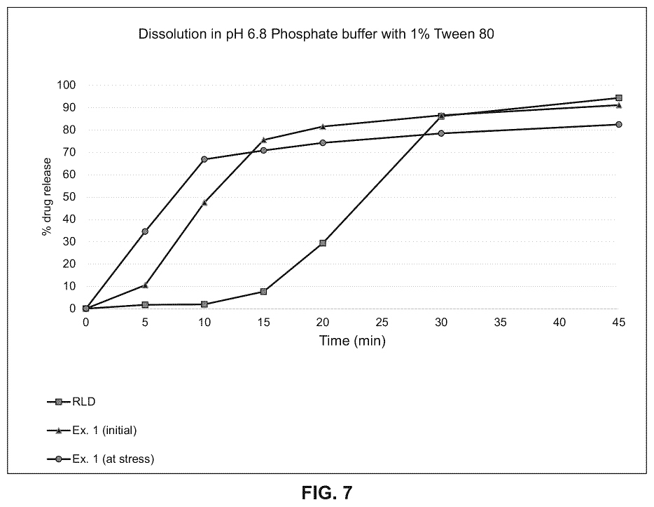

TABLE 1
Dissolution data of pharmaceutically acceptable formulation
of Tafamidis hard gelatin 61 mg capsule prepared by present
invention as per the Example 1, also shown in FIG. 7:

| | Product | | | | | |
|---|---|---|---|---|---|---|
| | | | | | Inventive | |
| | | | | | Formulation | |
| | | | Inventive | | (at stress condition: | |
| | | | Formulation | | 50° C., 1 week, | |
| | RLD-VYNDAMAX | | (Initial) | | open) | |
| | Media | | | | | |
| | 0.05M phosphate buffer pH 6.8 with 1% Tween 80, 900 ml, | | | | | |
| | Paddle with sinker, 75 RPM, (temperature: 37 ± 0.5° C.) | | | | | |
| Time | % | % | % | % | % | % |
| (min) | Release | RSD | Release | RSD | Release | RSD |
| 5 | 1.7 | 61.6 | 10.5 | 35.9 | 34.5 | 24.2 |
| 10 | 1.9 | 59.4 | 47.5 | 1.4 | 66.8 | 4.7 |
| 15 | 7.6 | 103.5 | 75.5 | 2.1 | 70.8 | 2.8 |
| 20 | 29.3 | 123.7 | 81.5 | 2.8 | 74.2 | 2.5 |
| 30 | 86.1 | 5.8 | 86.5 | 2.3 | 78.4 | 1.7 |
| 45 | 94.3 | 1.3 | 91.1 | 2.1 | 82.4 | 1.7 |

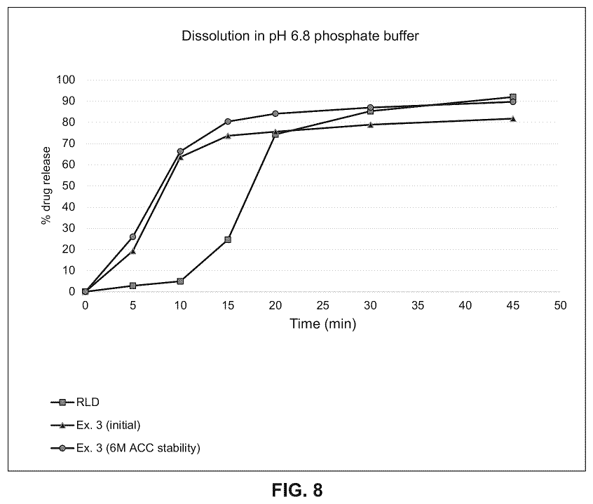

TABLE 2
Dissolution data of pharmaceutically acceptable formulation of
Tafamidis meglumine hard gelatin 20 mg capsule prepared by present
invention as per the Example 3, also shown in FIG. 8:

| | Product | | | | | |
|---|---|---|---|---|---|---|
| | | | | | Inventive | |
| | | | | | Formulation | |
| | | | | | (at accelerated | |
| | | | | | condition | |
| | | | Inventive | | 40° C. ± 2° C./ | |
| | | | Formulation | | 75% RH ± 5% RH, | |
| | RLD-VYNDAQEL | | (Initial) | | 6 Months) | |
| | Media | | | | | |
| | 0.05M phosphate buffer pH 6.8, 900 ml, | | | | | |
| | Paddle with sinker, 75 RPM (temperature: 37 ± 0.5° C.) | | | | | |
| Time | % | % | % | % | % | % |
| (min) | Release | RSD | Release | RSD | Release | RSD |
| 5 | 2.8 | 56.2 | 19.2 | 13.2 | 25.9 | 27.2 |
| 10 | 4.9 | 44.5 | 63.5 | 10.5 | 66.2 | 4.4 |
| 15 | 24.5 | 52.4 | 73.6 | 3.0 | 80.3 | 2.4 |
| 20 | 74.2 | 10.3 | 75.5 | 2.4 | 84.0 | 2.3 |
| 30 | 85.2 | 2.7 | 78.8 | 2.6 | 86.9 | 2 |
| 45 | 91.9 | 4.3 | 81.7 | 2.9 | 89.6 | 2.2 |

TABLE 3
Dissolution data of pharmaceutically acceptable
formulation of Tafamidis 61 mg capsule prepared
by present invention as per Example 4 and RLD:

| | Product | | | | | |
|---|---|---|---|---|---|---|
| | | | | | Inventive | |
| | | | | | Formulation | |
| | | | | | (at accelerated | |
| | | | | | condition | |
| | | | Inventive | | 40° C. ± 2° C./ | |
| | | | Formulation | | 75% RH ± 5% RH, | |
| | RLD-VYNADAMAX | | (Initial) | | 1 Month) | |
| | Media | | | | | |
| | 0.05M phosphate buffer pH 6.8 with 1% tween 80, 900 ml, | | | | | |
| | Paddle with sinker, 75 RPM (temperature: 37 ± 0.5° C.) | | | | | |
| Time | % | % | % | % | % | % |
| (min) | Release | RSD | Release | RSD | Release | RSD |
| 5 | 1.7 | 61.6 | 6.0 | 17.1 | 39.4 | 26.6 |
| 10 | 1.9 | 59.4 | 28.9 | 14.9 | 63.7 | 1.70 |
| 15 | 7.6 | 103.5 | 50.9 | 7.9 | 73.1 | 2.3 |
| 20 | 29.3 | 123.7 | 67.1 | 4.1 | 78.0 | 2.9 |
| 30 | 86.1 | 5.8 | 85.0 | 0.1 | 83.9 | 1.8 |
| 45 | 94.3 | 1.3 | 94.4 | 2.0 | 88.5 | 1.3 |

TABLE 4
Dissolution data of pharmaceutically acceptable
formulation of Tafamidis 61 mg capsule prepared
by present invention as per Example 5 and RLD:

| | | | Inventive Formulation | |
|---|---|---|---|---|
| | RLD-VYNDAMAX | | (Initial) | |
| Product | 0.05M phosphate buffer pH 6.8 with 1% tween 80, 900 ml, | | | |
| Media | Paddle with sinker, 75 RPM (temperature: 37 ± 0.5° C.) | | | |
| Time (min) | % Release | % RSD | % Release | % RSD |
| 5 | 1.7 | 61.6 | 4.9 | 6.3 |
| 10 | 1.9 | 59.4 | 25.3 | 9.2 |
| 15 | 7.6 | 103.5 | 47.4 | 9.4 |
| 20 | 29.3 | 123.7 | 66.3 | 6.7 |
| 30 | 86.1 | 5.8 | 86.7 | 4.5 |
| 45 | 94.3 | 1.3 | 93.8 | 4.0 |

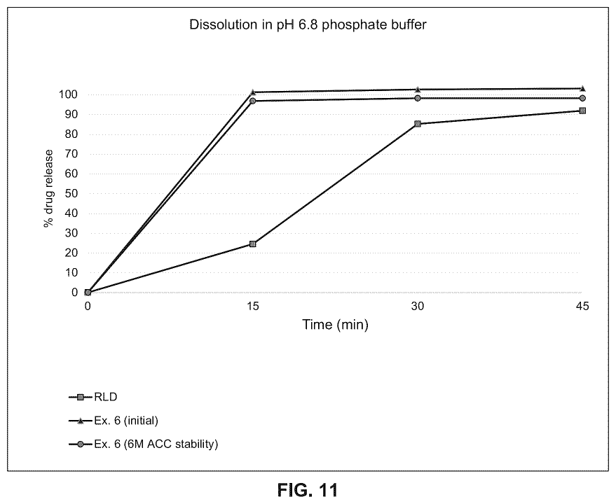

TABLE 5
Dissolution data of pharmaceutically acceptable formulation
of Tafamidis meglumine 20 mg capsule prepared by present invention
as per the Example 6 and RLD, also shown in FIG. 11:

| | Product | | | | | |
|---|---|---|---|---|---|---|
| | | | | | Inventive | |
| | | | | | Formulation | |
| | | | | | (at accelerated | |
| | | | | | condition | |
| | | | Inventive | | 40° C. ± 2° C./ | |
| | | | Formulation | | 75% RH ± 5% RH, | |
| | RLD-VYNDAQEL | | (Initial) | | 6 Month) | |
| | Media | | | | | |
| | 0.05M phosphate buffer pH 6.8 with 1% tween 80, 900 ml, | | | | | |
| | Paddle with sinker, 75 RPM (temperature: 37 ± 0.5° C.) | | | | | |
| Time | % | % | % | % | % | % |
| (min) | Release | RSD | Release | RSD | Release | RSD |
| 15 | 24.5 | 52.4 | 101.2 | 1 | 96.8 | 1.2 |
| 30 | 85.2 | 2.7 | 102.6 | 0.9 | 98.2 | 1.5 |
| 45 | 91.9 | 4.3 | 103.1 | 0.5 | 98.2 | 1.2 |

The inventive formulations exhibit comparable dissolution at 6-month accelerated storage conditions.

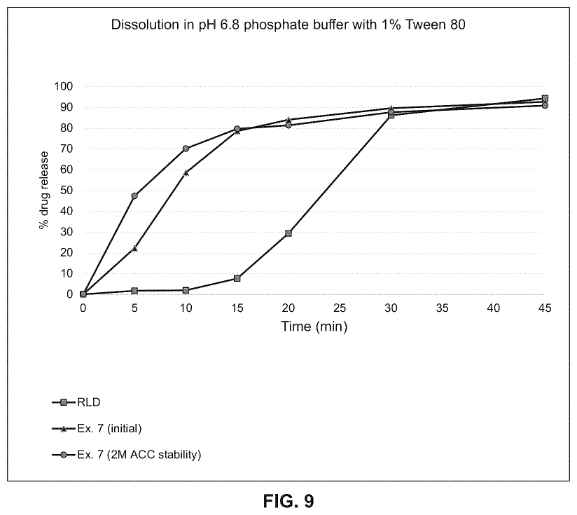

TABLE 6
Dissolution data of pharmaceutically acceptable formulation
of Tafamidis 61 mg capsule prepared by present invention
as per Example 7 and RLD, also shown in FIG. 9:

| | Product | | | | | |
|---|---|---|---|---|---|---|
| | | | | | Inventive | |
| | | | | | Formulation | |
| | | | | | (at accelerated | |
| | | | | | condition | |
| | | | Inventive | | 40° C. + 2° C./ | |
| | | | Formulation | | 75% RH ± 5% RH, | |
| | RLD-VYNDAMAX | | (Initial) | | 2 Month) | |
| | Media | | | | | |
| | 0.05M phosphate buffer pH 6.8 with 1% tween 80, 900 ml, | | | | | |
| | Paddle with sinker, 75 RPM (temperature: 37 ± 0.5° C.) | | | | | |
| Time | % | % | % | % | % | % |
| (min) | Release | RSD | Release | RSD | Release | RSD |
| 5 | 1.7 | 61.6 | 22.2 | 29 | 47.3 | 5.4 |
| 10 | 1.9 | 59.4 | 58.5 | 9 | 70.1 | 1.9 |
| 15 | 7.6 | 103.5 | 78.5 | 3.1 | 79.6 | 1.4 |
| 20 | 29.3 | 123.7 | 84 | 2.6 | 81.3 | 1.8 |
| 30 | 86.1 | 5.8 | 89.5 | 2.1 | 87.6 | 0.9 |
| 45 | 94.3 | 1.3 | 92.6 | 1.3 | 90.8 | 1.6 |

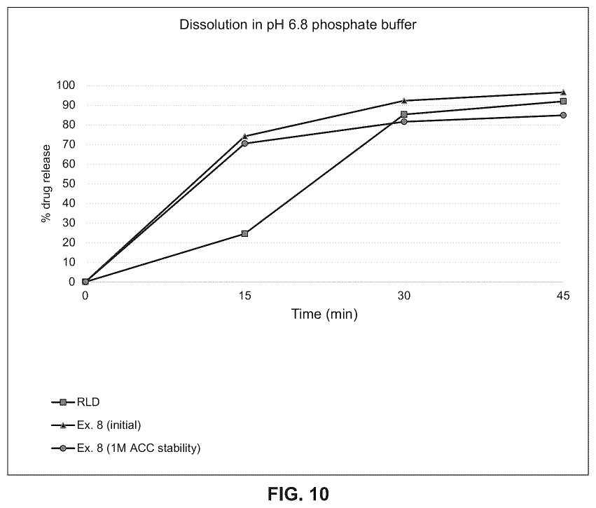

TABLE 7
Dissolution data of pharmaceutically acceptable formulation
of Tafamidis meglumine 80 mg capsule prepared by present invention
as per the Example 8 and RLD, also shown in FIG. 10:

| | Product | | | | | |
|---|---|---|---|---|---|---|
| | | | | | Inventive | |
| | | | | | Formulation | |
| | | | | | (at accelerated | |
| | | | | | condition | |
| | | | Inventive | | 40° C. ± 2° C./ | |
| | RLD-VYNDAQEL | | Formulation | | 75% RH ± 5% RH, | |
| | (20 mg) | | (Initial) | | 1 Month) | |
| | Media | | | | | |
| | 0.05M phosphate buffer pH 6.8 with 1% tween 80, 900 ml, | | | | | |
| | Paddle with sinker, 75 RPM (temperature: 37 ± 0.5° C.) | | | | | |
| Time | % | % | % | % | % | % |
| (min) | Release | RSD | Release | RSD | Release | RSD |
| 15 | 24.5 | 52.4 | 74.1 | 13.91 | 70.4 | 8.5 |
| 30 | 85.2 | 2.7 | 92.2 | 1.97 | 81.5 | 2.8 |
| 45 | 91.9 | 4.3 | 96.5 | 2.22 | 84.8 | 2.8 |

TABLE 8
Dissolution data of pharmaceutically acceptable formulation
of Tafamidis 61 mg capsule prepared by present invention
as per Example 9, Example 10, and RLD:

| | Product | | | | | |
|---|---|---|---|---|---|---|
| | | | Inventive | | | |
| | | | Formulation | | Formulation | |
| | | | as per | | as per | |
| | | | Example 9 | | Example 10 | |
| | RLD-VYNDAMAX | | (Initial) | | (Initial) | |
| | Media | | | | | |
| | 0.05M phosphate buffer pH 6.8 with 1% tween 80, 900 ml, | | | | | |
| | Paddle with sinker, 75 RPM (temperature: 37 ± 0.5° C) | | | | | |
| Time | % | % | % | % | % | % |
| (min) | Release | RSD | Release | RSD | Release | RSD |
| 10 | 1.9 | 59.4 | 72.4 | 19.63 | 55.8 | 2.7 |
| 15 | 7.6 | 103.5 | 80.2 | 8.63 | 62.6 | 0.9 |
| 20 | 29.3 | 123.7 | 82.3 | 7.94 | 67.1 | 2.8 |
| 30 | 86.1 | 5.8 | 85.8 | 7.00 | 73.8 | 1.3 |
| 45 | 94.3 | 1.3 | 89.4 | 6.16 | 79.6 | 1.2 |

Inventors of the present invention have observed that the formulation without cyclodextrin exhibits slower dissolution than the formulation containing cyclodextrin.

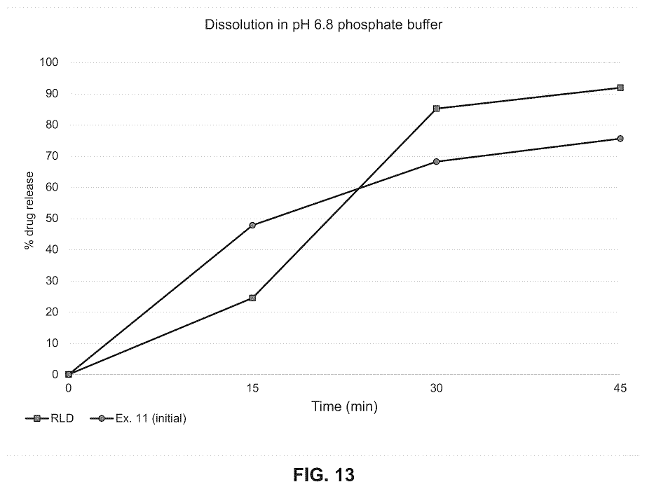

TABLE 9
Dissolution data of pharmaceutically acceptable formulation
of Tafamidis meglumine 80 mg capsule prepared by present invention
as per the Example 11 and RLD, also shown in FIG. 13:

| | RLD-VYNDAQEL | Inventive Formulation |
|---|---|---|
| | (20 mg) | (Initial) |
| Product | 0.05M phosphate buffer pH 6.8 with 1% tween 80, 900 ml, | |
| Media | Paddle with sinker, 75 RPM (temperature: 37 ± 0.5° C.) | |
| Time (min) | % Release | % Release |
| 15 | 24.5 | 47.8 |
| 30 | 85.2 | 68.2 |
| 45 | 91.9 | 75.6 |

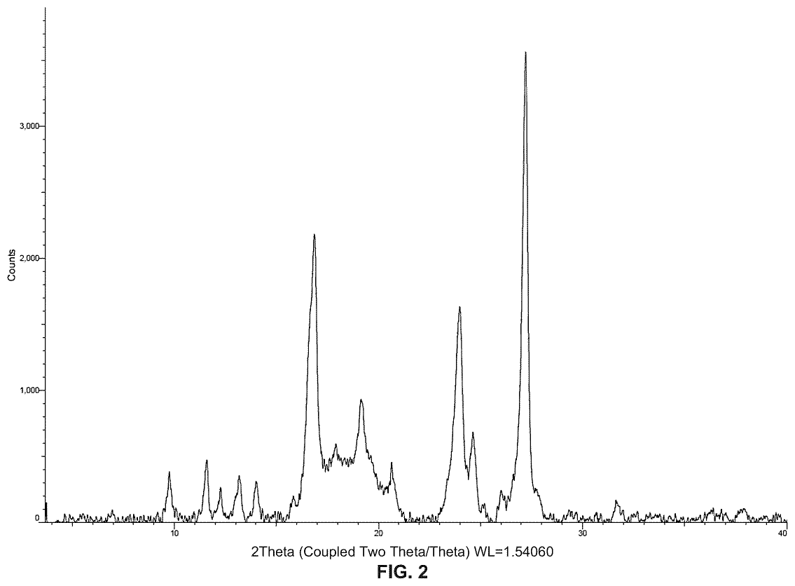

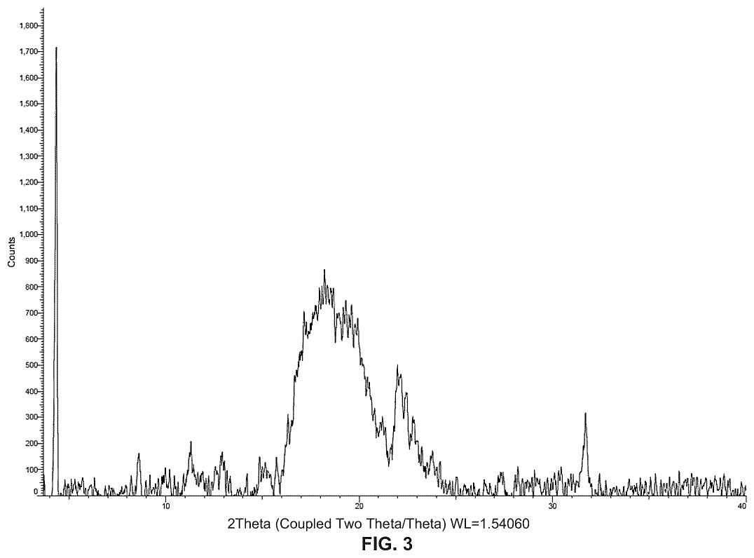

TABLE 10
XRPD analysis results of pharmaceutically acceptable
formulation of Tafamidis hard gelatin capsule prepared
by present invention as per the Example 1:

| Characteristic peak | Characteristic peak (2θ) | |
|---|---|---|
| (2θ) of original | from formulation | Placebo formulation |
| polymorphic form of | (at stress condition: | (at stress condition: |
| Tafamidis API | 50° C., 1 week, open) | 50° C., 1 week, open) |
| (FIG. 1) | (FIG. 2) | (FIG. 3) |
| 15.9 | 15.8 | — |
| 16.9 | 16.9 | — |
| 18.0 | 17.9 | — |
| 24.1 | 24.0 | 22.0 |
| 27.3 | 27.2 | 31.7 |

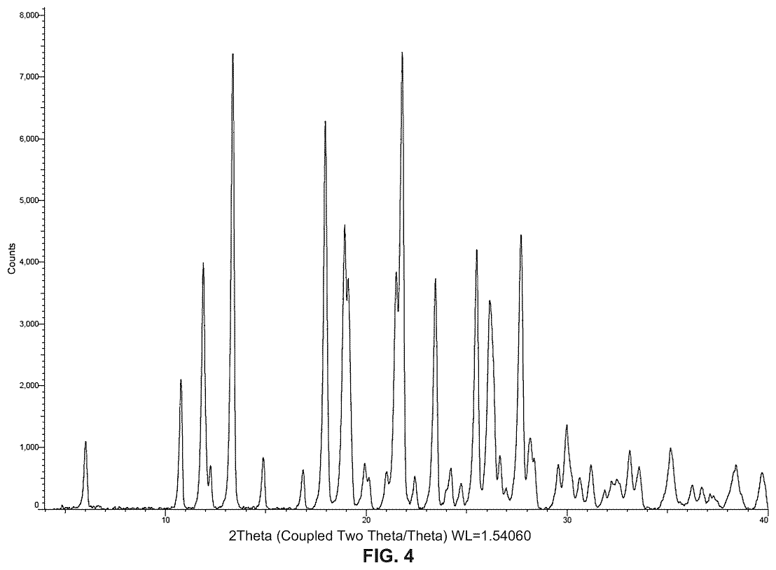

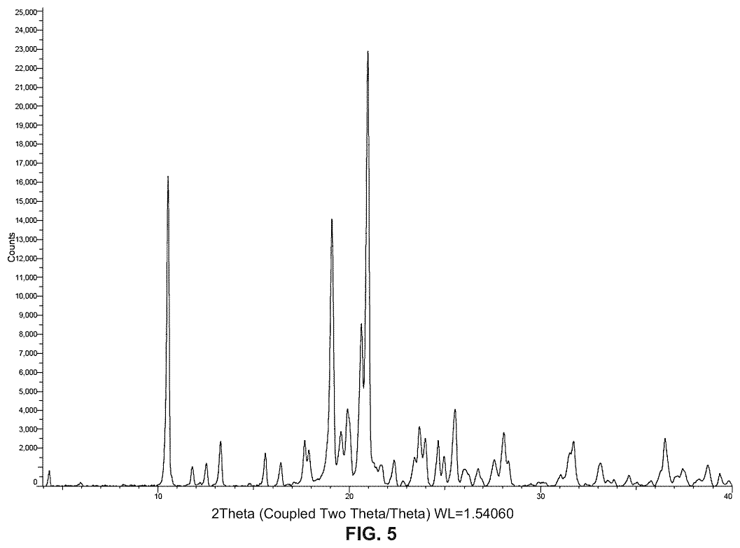

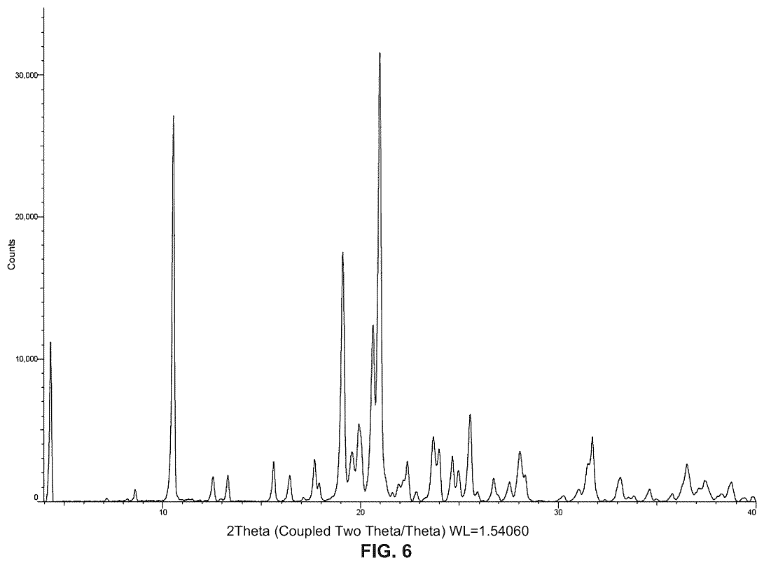

TABLE 11
XRPD analysis results of pharmaceutically acceptable
formulation of Tafamidis meglumine hard gelatin capsule
prepared by present invention as per the Example 3:

| Characteristic peak | Characteristic peak (2θ) | |
|---|---|---|
| ((2θ) of original | from formulation (at | Placebo formulation |
| polymorphic | accelerated | (at accelerated |
| form of | condition | condition 40° C. ± |
| Tafamidis | 40° C. ± | 2° C./75% |
| meglumine | 2° C./75% RH ± | RH ± 5% RH, |
| API | 5% RH, 3 Month) | 3 Month) |
| (FIG. 4) | (FIG. 5) | (FIG. 6) |
| 10.7 | 10.5 | 10.5 |
| 11.8 | 11.8 | 11.5 |
| 13.3 | 13.3 | 12.9 |
| 14.8 | 14.8 | 15.6 |
| 21.7 | 21.6 | 22.2 |

It should be noted that the invention in its broader aspects is not limited to the specific details, representative compositions, methods, and processes, and illustrative examples described in connection with the preferred embodiments and preferred methods. Modifications and equivalents will be apparent to practitioners skilled in this art and are encompassed within the spirit and scope of the appended claims.

What is claimed is:

1. An oral pharmaceutically acceptable formulation comprising:
an effective amount of at least one of tafamidis, a pharmaceutically acceptable salt thereof, and a tafamidis co-crystal, having a D90 particle size of about 20 microns or less;
at least one solubilizing agent selected from cyclodextrins, substituted cyclodextrin derivatives, and mixtures thereof; and
at least one excipient selected from diluent, disintegrant and lubricant;
wherein the formulation is provided in a form of a powder filled hard gelatin capsule.

2. The formulation according to claim 1, wherein the effective amount of tafamidis is about 61 mg per capsule.

3. The formulation according to claim 1, wherein the pharmaceutically acceptable salt of tafamidis is tafamidis meglumine in an amount of about 20 mg per capsule.

4. The formulation according to claim 1, wherein the pharmaceutically acceptable salt of tafamidis is tafamidis meglumine in an amount of about 80 mg per capsule.

5. The formulation according to claim 1, wherein the tafamidis co-crystal is selected from fumaric acid, adipic acid, glutaric acid, and a mixture thereof.

6. The formulation according to claim 5, wherein the tafamidis co-crystal is tafamidis fumaric acid.

7. The formulation according to claim 1, wherein the effective amount of tafamidis co-crystal is equivalent to about 61.0 mg of tafamidis about per capsule.

8. The formulation according to claim 1, wherein the at least one solubilizing agent is selected from cyclodextrins, substituted cyclodextrin derivatives, and mixtures thereof.

9. The formulation according to claim 8, wherein the at least one solubilizing agent is hydroxypropyl β-cyclodextrin.

10. The formulation according to claim 1, wherein the at least one solubilizing agent is provided in a range from about 10 wt. % to about 75 wt.

11. The formulation according to claim 10, wherein the at least one solubilizing agent is provided in a range from about 25 wt. % to about 40 wt.

12. The formulation according to claim 1, wherein the at least one diluent is selected from lactose, pregelatinized starch, corn starch, microcrystalline cellulose, mannitol, dicalcium phosphate, and mixtures thereof.

13. The formulation according to claim 1, wherein the at least one diluent is provided in a range from about 12 wt. % to about 40 wt. %.

14. The formulation according to claim 1, wherein the at least one disintegrant is selected from sodium starch glycolate, crospovidone, croscarmellose sodium, and mixtures thereof.

15. The formulation according to claim 1, wherein the at least one disintegrant is provided in a the range from about 1 wt. % to about 3 wt. %.

16. The formulation according to claim 1, wherein the at least one lubricant is selected from sodium stearyl fumarate, talc, magnesium stearate, and mixtures thereof.

17. The formulation according to claim 1, wherein the at least one lubricant is provided in a range from about 1 wt. % to about 2 wt. %.

18. The formulation according to claim 1, wherein tafamidis or the pharmaceutically acceptable salt thereof has a D90 particle size of about 20 microns or less.

19. An oral pharmaceutically acceptable formulation comprising:
an effective amount of at least one of tafamidis, a pharmaceutically acceptable salt thereof, and a tafamidis co-crystal;
at least one solubilizing agent; and
at least one excipient selected from diluent, disintegrant and lubricant;
wherein the formulation is provided in a form of a hard gelatin capsule.

20. A method of manufacturing the pharmaceutically acceptable formulation of claim 19, comprising:
providing an effective amount of the at least one of tafamidis, the pharmaceutically acceptable salt thereof, and the tafamidis co-crystal, in a dry form;
mixing the dry tafamidis, the dry pharmaceutically acceptable salt thereof, or the dry tafamidis co-crystal with the at least one solubilizing agent in a dry form;
adding the at least one excipient selected from diluent, disintegrant and lubricant in a dry form and mixing; and
encapsuling resulting mass in a hard gelatin capsule.

21. The method of claim 20, wherein the formulation is essentially free of water or another solvent during the entire manufacturing method.

22. A method of treating a transthyretin amyloid disease in mammals, comprising administering a therapeutically effective amount of the pharmaceutically acceptable capsule formulation of claim 19 to a mammal in need thereof.
